(19) United States
(12) Patent Application Publication
(10) Pub. No.: US 2004/0073278 A1
(43) Pub. Date: Apr. 15, 2004

(54) Method of and device for therapeutic illumination of internal organs and tissues

(76) Inventor: Freddy Pachys, Jerusalem (IL)

Correspondence Address: Anthony Castorina, G E Ehrlich, Suite 207, 2001 Jefferson Davis Highway, Arlington, VA, 22202, US

(21) Appl. No.: 10/466,776

(22) PCT Filed:
(86) PCT No.: PCT/IL02/00731

(51) Int. Cl. A61N 1/00

(52) U.S. Cl. 607/88; 607/94

(57) ABSTRACT

Methods and devices for intracorporeal therapeutic illumination using an implantable light source are disclosed. The present invention can be used for short, intermediate and/or long-term light therapy of all internal tissues, organs and organ surfaces, including the blood, in the treatment of inflammatory, infectious, arthritic, allergic, musculoskeletal and parasitic pathologies. The implantable light source comprises a wide range of modifiable wavelengths and other light therapy parameters and can illuminate remote intracorporeal locations via optional fiber optic connection. Direct phototherapy of the blood is effected by implantable intravascular light sources, optical fiber conduits, a unique light source-bearing intravascular tubular platform or a novel, light emitting vascular prosthesis. Power and modulation of the light therapy parameters is provided by implantable power and control modules or, in preferred embodiments, by external sources in telemetric communication with the implanted light source. Light therapy parameters for individual treatment protocols can be precisely modulated in response to the physiological status of the patient, feedback from the tissues being treated or other, external stimuli.

### FIELD AND BACKGROUND OF THE INVENTION

The present invention relates to a method of and device for providing therapeutic photostimulation, also referred to herein as therapeutic illumination, to internal organs and tissues, including the blood, via an intracorporeally implanted light source.

General

Light energy is commonly employed in medicine for a variety of therapeutic purposes. Target tissues and/or molecules capable of absorbing a portion, or all, of the energy available in the light reaching them may be modified and/or stimulated to achieve substantial changes in morphological, biochemical or metabolic properties. Appropriately and carefully applied, such photostimulation has been shown to be beneficial for many local and systemic conditions.

Ultraviolet Therapy and UV Blood Irradiation

One such application is the irradiation of blood and other body fluids with wavelengths in the ultra violet (UV) range (<400 nm), pioneered in the early 20^{th} century by Knott (U.S. Pat. Nos. 2,308,516 and 2,309,124 to Knott). Following specific protocols proposed by the American Blood Irradiation Society, therapists using external UV irradiation of whole blood aliquots have achieved positive results in the treatment of infectious conditions such as a typical pneumonia, poliomyelitis and polioencephalitis, hepatitis, HIV, AIDS, influenza, common upper respiratory diseases, herpes simplex and zoster, mumps, mononucleosis and measles (For a brief review see U.S. Pat. No. 6,113,556 to Schleicher). Treatment of chronic conditions such as rheumatoid arthritis (Zonova E B, Prokof'ef V F, Ivanova R L and Konenkov V I. Immunogenic methods in the prognosis of the efficacy of using a method of transfusing extracorporeally irradiated autologous blood for treating patients with rheumatoid arthritis. Gematol. Transfuziol. 1993 38(2): 33-36) atherosclerosis (Adamchik A S, Sushkevich G N, Kubateiv A A and Belov I V. The antithrombogenic properties of the vascular wall and platelet aggregation in patients with atherosclerosis of the arteries of the lower extremeties following a course of treatment with UV-irradiated autologous blood transfusion. 1993 38(2): 23-26) and endotoxic syndrome in bronchial asthma (Tkachenko I L. The effect of UV irradiation of the blood and of hemosorption of the biochemical signs of endogenous intoxication in asthma patients. 1999 Lik. Sprava June(4) 121-24) have also been reported. The specific beneficial effects of UV blood therapy seem to be associated with an increase in oxygenation of the blood, stimulation of endogenic antioxidant production, increased phagocytosis and reduction of edema, toxemia, nausea and vomiting.

Ultraviolet irradiation of skin surfaces has long been recognized as effective in treatment of infectious and metabolic disorders of the skin and underlying dermal layers. Indeed, UV exposure is the most often prescribed mode of therapy for neonatal hyperbilirubunemia and porphyria.

Visible and Infrared Spectrum Light Therapy

Longer wavelength light, of the visible and infrared portion of the electromagnetic wave spectrum, has also been used therapeutically. U.S. Pat. Nos. 6,156,028 and 5,616,140 to Prescott describe illumination with low level laser radiation in the range of 400-1,300 nm for enhancing the healing of leg ulcers, preventing osteomyelitis and improving circulation in diabetics, for relief of joint stiffness and pain control in arthritics, for reduced scarring and duration of healing in fractures, stimulation of neurotransmitters, endocrine function and modulation of the immune system via T-cell, B-cell and leukocyte activity. Similarly, U.S. Pat. No. 5,259,380 to Mendes et al. describes illumination with low power non-coherent light of red and infra-red wavelength for biostimulation and healing of skin ulcers and delayed postoperative wound healing. Many dermatological conditions, including psoriasis and acne are commonly treated by a variety of regimens of phototherapy (Horio T. Indications and action mechanisms of Phototherapy. 2000 J. Dermatol Sci March 23 Suppl 1: S12-21), and allergic rhinitis and nasal polyposis have been treated with 660 nm laser light (Neumann I and Finkelstein Y. Narrow-band red light phototherapy in perennial allergic rhinitis and nasal polyposis. 1997 Ann Allergy Asthma Immunol April; 78(4) 399-406).

Non-surgical, low level laser therapy is thought to effect numerous metabolic processes, including cell division, cyclic-AMP metabolism, oxidative phosphorylation, hemoglobin, collagen and other protein synthesis, leukocyte activity, tumor growth, production of macrophage cells and wound healing. See, for example, Karu and Letokhov “Biological Action of Low-Intensity Monochromatic Light in the Visible Range” in Laser Photobiology and Photomedicine, ed. Martellucci, p. 57-66 (Plenum Press 1985); Passarella, et al., “Certain Aspects of Helium-Neon Laser Irradiation on Biological Systems in Vitro” in Laser Photobiology and Photomedicine, ed. Martellucci p. 67-74 (Plenum Press 1985); see generally, Parrish, “Photomedicine: Potentials for Lasers. An Overview,” in Lasers in Photomedicine and Photobiology, ed. Pratesi, p. 2-22 (Springer 1980); Giese, “Basic Photobiology and Open Problems” in Lasers in Photomedicine and Photobiology, ed. Pratesi, p. 26-39 (Springer 1980); Jori, “The Molecular Biology of Photodynamic Action” in Lasers in Photomedicine and Photobiology, ed. Pratesi, p. 58-66 (Springer 1980).

Although the precise mechanism for these effects is not fully understood, it is believed to be associated with the activity of specific wavelengths of radiation in or near the range of visible light. Infrared laser radiation has been shown to increase ATP concentration and ATPase activity in living tissues (Bolognani, et al., “Effects of GaAs Pulsed Lasers on ATP Concentration and ATPase Activity In Vitro and In Vivo”, International Cong. on Lasers in Medicine and Surgery, p. 47 (1985).

Seasonal Affective Disorder (SAD), bullemia, “jet lag”, shift work sleep disturbance and other misalignments of circadian rhythm have also been treated with phototherapy. Whereas the benefits of high intensity, visible spectrum illumination in the treatment of these conditions were previously thought to depend on activation of ocular photosensors, the phenomenon of non-ocular response to phototherapy is now widely accepted (Parker J S, Flory R K, Everhart D E and Denrow D M. Casereport: Neurochemical, physiological and behavioral effects of bright light therapy on a cortically blind patient. 1996 Int. J. Neurosci December 88(3-4) 273-82; and U.S. Pat. No. 6,135,117 to Campbell et al.).

Photodynamic Therapy and Intracorporeal Illumination

Traditional methods of phototherapy have depended upon the application of light energy from outside the body. Numerous and varied protocols of extracorporeal illumination of tissue surfaces exist for phototherapy of both surface structures and tissue components, and of deeper photosensitive elements. Thus, extracorporeal illumination with low level laser light is used to treat not only inflammations, wounds, burns, chronic ulcerations, eczema, shingles, infection, scars, skin grafts, gingival irritation, oral ulcers, cellulitis, stretch marks, skin tone and alopecia areata (see, for example, U.S. Pat. No. 4,930,504 to Diamantopolous et al.), but also arthritic conditions such as chondromalacia patellae, facet joint arthritis, tendinitis (U.S. Pat. No. 5,259,380 to Mendes et al.), muscle pain and stiffness, myofascial pain; post surgical complications, such as swelling, inflammation, scarring and stiffness; acute trauma and chronic post-traumatic conditions in the soft tissues and bones, including sprains, strains, wounds, whiplash; repetitive strain injuries such as carpal tunnel syndrome, tennis and golfer's elbow; neurological and neuromuscular conditions (U.S. Pat. No. 6,063,108 to Salansky et al.). Typical protocols employ manipulation of pulse width and repetition frequency, wavelength, bandwidth, intensity and density of the illumination using directly or remotely coupled power sources, control modules and light emitting elements.

Another, widely used application of phototherapy is the photoactivation of therapeutic compounds, known as PhotoDynamic Therapy, or PDT. Abnormal cells in the body are known to selectively absorb certain dyes perfused into a treatment site to a much greater extent than surrounding tissue. For example, tumors of the pancreas and colon may absorb two to three times the volume of certain dyes, compared to normal cells. Once pre-sensitized by dye tagging, the cancerous or abnormal cells can be destroyed by irradiation with light of an appropriate wavelength or waveband corresponding to an absorbing wavelength or waveband of the dye, with minimal damage to normal tissue. PDT has been clinically used to treat metastatic breast cancer, bladder cancer, lung carcinomas, esophageal cancer, basal cell carcinoma, malignant melanoma, ocular tumors, head and neck cancers, and other types of malignant tumors (see, for example, U.S. Pat. No. 5,800,478 to Chen et al.).

However effective extracorporeal illumination for phototherapy of internal tissues may be, the method suffers from the inherent disadvantages of absorption of light energy by layers of complex overlying tissue, leading to imprecise control of therapeutic parameters such as pulse frequency, intensity and wavelength; poor localization of target surfaces, due to scatter and thermal effects; and the sometimes massive, unintentional collateral irradiation of healthy tissue. In addition, the traditionally bulky and expensive equipment required for extracorporeal phototherapy precluded long-term exposure of target tissue, restricting therapy to clinical location and schedules. Thus, various solutions employing internal, or intracorporeal illumination have been proposed.

One such approach to intracorporeal illumination is via a catheter or endoscope. U.S. Pat. No. 5,693,049 to Mersch describes a catheter comprising a light emitting surface, or light emitting element enclosed within a transparent sheath, introduced into vascular elements for therapeutic illumination of the blood in vivo. Fiber-optic transmission of light within an endoscopic catheter is described by Doiron et al (U.S. Pat. No. 5,728,092) for illumination and phototherapy of hollow organs such as the bladder, stomach, colon, heart, esophagus, etc. Prescott (U.S. Pat. No. 6,156,028) describes phototherapy, with low level laser illumination, of lumen surfaces using an flexible, light emitting probe and an optically clear balloon catheter, for healing vascular tissue in angioplasty procedures and following vascular graft surgery. Also described is an array of light emitting diodes mounted on the surface of a needle catheter, for illumination of dense and solid tissue, and the placement of flexible light emitting probes around a body part or organ for internal phototherapy. However, such devices are intended to provide illumination for a limited period only, as they are introduced in the course of an endoscopic or surgical procedure, or transcutaneously, and are powered and controlled by external sources. No mention of an implantable, self-contained device for intracorporeal phototherapy is made.

Chen et al. (U.S. Pat. Nos. 5,445,608; 5,571,152; 5,800,478 and 5,997,569) describe a variety of implantable light emitting diodes for intracorporeal photodynamic therapy. The light emitting diodes, illuminating within the wavelengths absorbed by perfused photoactive agents, are mounted on flexible (U.S. Pat. No. 5,800,478) or rigid (U.S. Pat. No. 5,445,608) probes for implantation. Although direct illumination by light emitting diodes is stressed, the authors also propose the incorporation of an external light source and an implantable fiber optic light probe.

Miniaturization of electronic components has facilitated the implantation of a variety of power supply and control elements, most commonly recognized for treatment of cardiac arrhythmia (pacemakers), but also applicable to phototherapy devices. Thus, Chen et al. describe the use of an implantable battery, or external power pack with implanted electric connections; and external microwave, electromagnetic and/or infrared power wirelessly connected to implanted receivers electrically coupled to the light source. Control of the optical and temporal parameters of the phototherapy regimen(s) may be unmodifiable, determined prior to implantation; or variable, adjusted according to need via an internal or external command unit in direct or wireless electrical connection with the light source. In additional, the authors describe “physiological” control of therapy parameters by addition to the circuitry of sensor elements (heat, blood pressure and pulse, chemosensors, EEG, ECG, etc.) providing information regarding the status of the individual/system/tissue undergoing treatment. However, these devices and methods are intended exclusively for PDT of internal tissues in conjunction with perfused photoactive agents.

U.S. Pat. No. 5,571,152 to Chen et al. describes a microminiature light emitting bead controlled and powered by remote electromagnetic and/or radio frequency energy, for PDT. Such a miniature light source is concieveably injectable, easily and relatively non-invasibly introduced into tissue, hollow organs or even vascular elements. Thus, the authors propose, deep or inconveniently located tissue could be easily illuminated intracorporeally. However, such a freely circulating light source is susceptible to uncontrollable movement by blood fluid dynamics, and, of greater concern, capable of causing occlusion of critical small blood vessels with serious medical consequences.

Thus, there is a widely recognized need for, and it would be highly advantageous to have, a controlled, implantable device and method for non-photodynamic phototherapy of blood and other internal tissues.

### SUMMARY OF THE INVENTION

According to one aspect of the present invention there is provided a method of therapeutic illumination of internal organs and/or tissues, the method comprising implanting intracorporeally in a subject in need of therapeutic illumination an implantable light source for producing light suitable for therapeutic illumination.

According to another aspect of the present invention there is provided a method of therapeutic illumination of blood, the method comprising implanting intracorporeally in a subject in need of therapeutic illumination an implantable light source for producing light suitable for therapeutic illumination of blood.

According to yet another aspect of the present invention there is provided a device for therapeutic illumination of internal organs and/or tissues, the device comprising a light source for producing light suitable for therapeutic illumination, a battery or energy transducer for powering the light source and at least one optical fiber in optical communication with the light source for propagating light emitted from the light source to a distant intracorporeal location, wherein the light source, the battery or energy transducer and the at least one optical fiber are designed and constructed for intracorporeal implantation.

According to still another aspect of the present invention there is provided a device for therapeutic illumination of a tissue and/or an organ, the device comprising an implantable vascular prosthesis capable of anastamosing connection to a blood vessel in which a lateral or terminal opening has been formed, a light source for producing light suitable for therapeutic illumination being optically connected to the vascular prosthesis and an implantable battery or energy transducer for powering the light source.

According to an additional aspect of the present invention there is provided a device for therapeutic illumination of blood, the device comprising an implantable vascular prosthesis capable of anastamosing connection to a blood vessel in which a lateral or terminal opening has been formed, the implantable vascular prosthesis having an internal light emitting surface for light irradiation of substances in fluid motion through the prosthesis.

According to yet an additional aspect of the present invention there is provided a device for therapeutic illumination of blood, the device comprising an implantable tubular platform allowing blood flow therethrough, a light source for producing light suitable for therapeutic illumination being carried by the implantable tubular platform and an implantable battery or energy transducer for powering the light source.

According to further features in preferred embodiments of the invention described below, the implantable light source is designed, constructed and implantable so as to illuminate a lumen of a blood vessel.

According to yet further features in the described preferred embodiments of the invention described below, the implantable light source is designed, constructed and implantable so as to illuminate a lumen of at least one heart chamber.

According to further features in the described preferred embodiments of the invention described below, the implantable light source is designed, constructed and implantable so as to illuminate a lumen of an organ, such as, for example, brain, spinal canal, sinuses, middle ear, lungs, esophagus, stomach, intestines, colon, pancreas, spleen, gall bladder, appendix, liver, kidney, bladder, heart, ovary and uterus.

According to yet further features in the described preferred embodiments of the invention described below, the implantable light source is designed, constructed and implantable so as to illuminate a surface of an internal organ, such as eye, brain, spinal cord, sinuses, middle ear, lungs, stomach, intestines, pancreas, spleen, liver, kidney, heart, ovary, uterus, testis, prostate, bladder, endocrine and/or exocrine glands, bone, muscle and connective tissue.

According to still further features in the described preferred embodiments of the invention described below, the light is a coherent light between 189 nm and 1,300 nm in wavelength.

According to yet further features in the described preferred embodiments of the invention described below, the light is a non-coherent light of a plurality of wavelengths and/or wavebands between 189 nm and 1,300 nm.

According to further features in the described preferred embodiments of the invention described below, the light is a non-coherent light of at least one waveband between 189 nm and 1,300 nm.

According to still further features in the described preferred embodiments of the invention described below, the tubular platform is transparent to light produced by the light source.

According to further features in the described preferred embodiments of the invention described below, the tubular platform is opaque to light produced by the light source.

According to still further features in the described preferred embodiments of the invention described below, the energy transducer is selected from the group consisting of a radiofrequency transducer, a magnetic transducer and an acoustic transducer.

According to further features in the described preferred embodiments of the invention described below, the implantable light source comprises and is powered by a battery or energy transducer integrally connected thereto.

According to yet further features in the described preferred embodiments of the invention described below, the energy transducer is selected from the group consisting of a radiofrequency transducer, a magnetic transducer and an acoustic transducer.

According to still further features in the described preferred embodiments of the invention described below, the implantable light source is powered by telemetry.

According to further features in the described preferred embodiments of the invention described below, the telemetry is selected from the group consisting of acoustic based telemetry, radiofrequency based telemetry and magnetic based telemetry.

According to yet further features in the described preferred embodiments of the invention described below, the implantable light source is controlled, the control comprising determining light therapy parameters selected from a group comprising dose, intensity, frequency, pulse duration, wavelength, power, monochromaticity, intensity modulation with specific endogenous frequencies and three dimensional photon distribution.

According to still further features in the described preferred embodiments of the invention described below, the light therapy parameters are preselected.

According to further features in the described preferred embodiments of the invention described below, the light therapy parameters are variably determined.

According to yet further features in the described preferred embodiments of the invention described below, the light therapy parameters are determined in respect to a physiological status of a subject being treated.

According to still further features in the described preferred embodiments of the invention described below, the physiological status is selected from the group consisting of EEG, EMG, ECG, blood chemistry, viral load, body temperature, chemiluminescence, pH, pulse and respiration.

According to further features in the described preferred embodiments of the invention described below, the implantable light source is controlled by telemetry, such as acoustic-based telemetry, radiofrequency-based telemetry or magnetic-based telemetry.

According to still further features in the described preferred embodiments of the invention described below, the implantable light source is controlled by an on-board logic-chip.

According to further features in the described preferred embodiments of the invention described below, the subject is treated for a pathology selected from the group consisting of inflammations, wounds, burns, chronic ulcerations, eczema, shingles, infection, scars, skin, vascular and organ grafts, gingival irritation, oral ulcers, cellulitis, arthritic conditions, muscle pain and stiffness, myofascial pain, swelling, inflammation, scarring and stiffness, sprains, strains, wounds, whiplash, repetitive strain injuries, neurological and neuromuscular conditions, jet lag, Seasonal Affective Disorder, shift work sleep disturbance, atheroslerosis following balloon angioplasty, allergic rhinitis and nasal polyposis.

According to yet further features in the described preferred embodiments of the invention described below, the subject is treated for a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

According to yet further features in the described preferred embodiments of the invention described below, the light source is a non-gaseous light emitting source.

According to still further features in the described preferred embodiments of the invention described below, the non-gaseous light source is selected form the group consisting of laser, light-emitting diodes, superluminous diodes and laser diodes.

According to further features in the described preferred embodiments of the invention described below, the at least one optical fiber is capable of adapting to the contour of body passages.

According to yet further features in the described preferred embodiments of the invention described below, the at least one optical fiber forms a bundle of optical fibers.

According to still further features in the described preferred embodiments of the invention described below, the bundle of optical fibers is flexible and hence capable of adapting to contours of body passages.

According to further features in the described preferred embodiments of the invention described below, the bundle of optical fibers is engaged within a sheath.

The present invention successfully addresses the shortcomings of the presently known configurations by providing a method and device for direct phototherapy of internal tissues, including blood. The resulting benefits include (i) intermediate and long term phototherapy of internal tissues; (ii) direct, long-term illumination of blood without effecting endothelium and neighboring tissues; (iii) provisions for intracorporeal and/or external (telemetric) power supply and control of illumination; and (iv) continuously variable, remote modulation of light therapy parameters.

Implementation of the method and device of the present invention involves performing or completing selected tasks or steps manually, automatically, or a combination thereof. Moreover, according to actual instrumentation and equipment of preferred embodiments of the method and device of the present invention, several selected steps could be implemented by hardware or by software or a combination thereof. For example, as hardware, selected steps of the invention could be implemented as a chip or a circuit. As software, selected steps of the invention could be implemented as a plurality of software instructions being executed by a computer using any suitable operating device. In any case, selected steps of the method and device of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic view of an implantable device for therapeutic illumination, with connected optical fiber illuminating the lumen of a blood vessel, in accordance with the teachings of the present invention;

FIG. 2 is a schematic view of the implantable device of FIG. 1, optically connected to an implantable light emitting vascular prosthesis, in accordance with the teachings of the present invention;

FIG. 3 is a cross-sectional view of the implantable light emitting vascular prosthesis, in accordance with the teachings of the present invention;

FIG. 4 is a schematic view of the implantable device of FIG. 2, with the implantable light emitting vascular prosthesis in terminal anastomosing connection with a vascular element;

FIG. 5 is a schematic view of the implantable device of FIG. 1, optically connected to an implantable tubular platform; and

FIG. 6 is a schematic view of the implantable device of FIG. 5, with the implantable tubular platform in place within the lumen of a blood vessel.

### DESCRIPTION OF THE PREFERRED EMBODIMENTS

The present invention is of a method and implantable device for intracorporeal therapeutic illumination of internal organs and tissues. Specifically, the present invention can be used for short, intermediate and/or long-term light therapy of all internal tissues, organs and organ surfaces, including the blood, in the treatment of inflammatory, infectious, arthritic, allergic, musculoskeletal and parasitic pathologies.

As used herein, the term pathology refers to any disease, syndrome, effect and/or medical condition which affects human health or well being.

The principles and operation of a method and implantable device for intracorporeal therapeutic illumination of internal organs and tissues, employing optical fibers according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Phototherapy is defined as the treatment of a disorder of a biological tissue by stimulation with light having selected optical parameters. Many applications of such therapeutic light irradiation are currently employed in medical practice, such as UV irradiation for hyperbilirubinemia and skin conditions (U.S. Pat. No. 4,930,504 to Diamantopolous et al.), high power laser irradiation for efficient and precise surgical procedures, low level laser irradiation for wound healing and relief of chronic inflammation (U.S. Pat. No. 5,259,380 to Mendes et al.), blood irradiation for infectious and toxic conditions (see U.S. Pat. No. 6,113,556 to Schleicher for a review), Photo Dynamic Therapy and light therapy for Seasonal Affective Disorder and disruptions of the circadian rhythm. It is clear, from these examples and others, that non-ocular responses to light stimulation are not only substantial, but also critical to both normal and pathological states.

Although natural light irradiation is provided by sources outside of the body (extracorporeal), it has become clear that the absorption of light energy by internal tissues is critical to the effectiveness of many phototherapeutic applications (see, for example, U.S. Pat. No. 4,930,504 to Diamontopolous et al., and U.S. Pat. No. 6,063,108 to Salansky et al.). Almost every mammalian cell may be photosensitive, e.g., could respond to light irradiation by changes in metabolism, reproduction rate or functional activity. Light photons are thought to be absorbed by some biological molecules, primary photoacceptors, presumably enzymes, inducing change in their biochemical activity. If enough molecules are affected by photons, this may trigger (accelerate) a complex cascade of chemical reactions to cause changes in cell metabolism. Light photons may just be a trigger for cellular metabolism regulation. This explains why low energies are sometimes adequate for these so called “photobiomodulation” phenomena.

However, reliance on extracorporeal illumination for photostimulation of deep tissues suffers from the inherent disadvantages of absorption of light energy by layers of complex overlying tissue, leading to imprecise control of therapeutic parameters such as pulse frequency, intensity and wavelength; poor localization of target surfaces, due to scatter and thermal effects; and the unintentional collateral irradiation of healthy tissue.

In order to avoid the abovementioned disadvantages, and to provide greater precision and efficiency of phototherapeutic stimulation, various methods of intracorporeal illumination have been proposed, for example, endoscopic illumination of blood vessels for cardiac surgery (U.S. Pat. No. 6,113,588 to Duhaylongsod et al.) and fiber optic catheters (U.S. Pat. No. 5,728,092 to Doiron et al.). Chen et al (U.S. Pat. Nos. 5,445,608; 5,997,569; 5,800,478 and 5,571,152) disclose elongated light emitting probes, flexible probes, implantable light emitting beads and other forms of intracorporeal light emitting devices for illumination of internal tissues for PhotoDynamic Therapy.

PhotoDynamic Therapy (PDT), employing perfusion of photosensitive dyes for the targeting of treatment to cancerous or otherwise diseased tissue by photoactivation, is distinguished from direct phototherapeutic stimulation of internal tissues in both technique and principle. Whereas PDT is indirect and essentially limited to the metabolically toxic effects of the photostimulated dyes on their target tissues, and the types of light radiation absorbed by these dyes, direct phototherapeutic stimulation of internal tissues incorporates all combinations of light parameters and is applicable to any and all tissues capable of absorbing light.

According to one aspect of the present invention there is provided a method of therapeutic illumination of internal organs and/or tissues, the method is effected by implanting intracorporeally in a subject in need of therapeutic illumination an implantable light source for producing light suitable for therapeutic illumination.

As used herein the phrase “light suitable for therapeutic illumination” refers to electromagnetic radiation, within the range of wavelengths between and inclusive of ultraviolet to infrared, capable of effecting a substantial change in the structure, function, biochemistry and/or metabolism of a viable tissue. It will be appreciated, in the context of the present invention, that the term “therapeutic” is not restricted to the treatment of a diseased or abnormal condition, but also includes all and any beneficial modulations of structure, function, biochemistry and/or metabolism of tissue or tissues, and/or of the organism undergoing treatment. Thus, the intracorporeal illumination of the present invention may be applied to enhance feed conversion, growth and/or milk production in cattle, for example, in addition to treatment of common inflammation and infection in such domestic species.

As used herein in the specification and in the claims section below, the term “implantable light source” refers to any source of electromagnetic radiation, within the range of wavelengths between and inclusive of ultraviolet to infrared, which may be surgically or transdermally inserted within an internal tissue, organ or cavity without substantially disrupting physiological function.

There are many types of light sources suitable for implantation. These include filament bulbs, gaseous and non-gaseous light sources. In one preferred embodiment of the present invention the light source is a non-gaseous light source, such as a laser, superluminous diode, laser diode, or, most preferably a light-emitting diode. The advantages of such light sources is their small size, wide range of wavelengths and bandwidths available, low energy demands per light output, relatively long life expectancy and minimal thermal output. Two particular types of LED's are most useful for purposes of the present invention: laser diodes and superluminous diodes. Laser diodes produce a beam of light or radiation that is essentially monochromatic, is sharply collimated and is coherent. That is, they produce light almost exclusively at one frequency (unless they are multi-mode type lasers) and the light beam has a small angle of divergence. Superluminous diodes are also used. These are similar but lack the coherence and the sharply monochromatic characteristics of laser diodes; yet they produce highly directional light that is also limited in its frequency range. A number of commercially available semiconductor laser diodes exist. Typical of these are those described in “Optoelectronic Devices Data Book” published by Hitachi, Ltd. (September, 1984).

Semiconductor laser diodes having somewhat higher power outputs and narrower beam divergence and spectral widths than the most widely manufactured components are also available and may enhance the advantages of the present invention. Not all frequencies are available in the range from ultraviolet through visible to infrared radiation. But enough are available that some selection among frequencies can be made. Among low power lasers suitable for the present invention, the laser power rating (continuous power) of individual diodes is generally in the range from 0.01-500 milliwatts (mW). Laser diodes are available with continuous wave emission capability and as devices that must be pulsed. Preferably, the light source is enclosed within biocompatible material which is optically transparent to at least one wavelength and/or at least one waveband.

The operation of the implantable light source requires a source of power and connection with the light source. A number of possible power supply options, and means of connection are available.

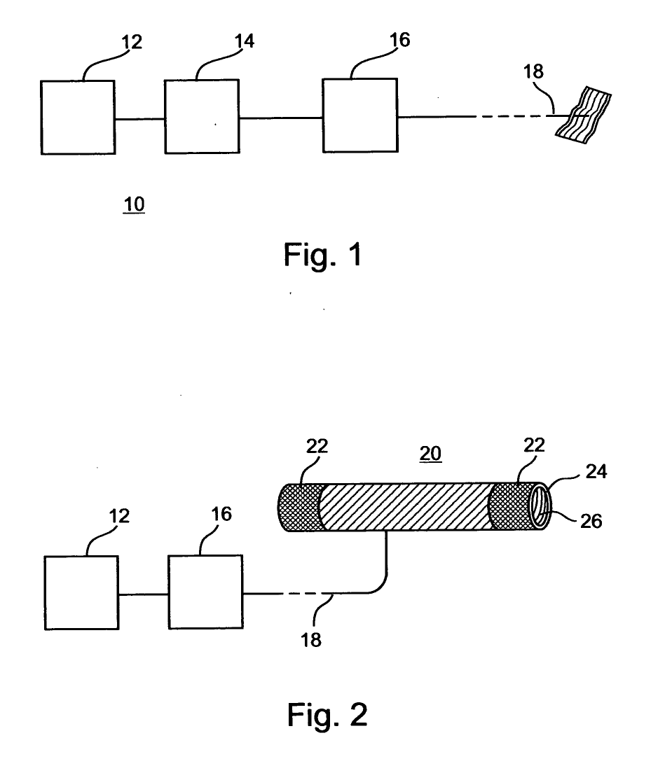

LEDs or other types of light source(s), and/or other types of micro-electronic circuits are provided electrical current to energize the devices through power leads from a power supply, which may comprise a battery mounted on/adjacent to the light source or at a remote site within the patient's body, or may be coupled electromagnetically, acoustically or through an RF signal, to an external source of power. FIG. 1 depicts an implantable device for therapeutic illumination 10, comprising power supply 12 for energizing light source 16, and control module 14 for determining optical parameters. In one embodiment of the present invention, as depicted in FIG. 1, light source 16 is in optical communication with optical fiber 18, which is depicted implanted into a blood vessel.

Implantable light source 16 may bear leads that extend from a remote, intracorporeal location and terminate in connectors for direct connection to power supply 12. However, as noted above, electrical power and signals can be conveyed between the light source and an external device, across a cutaneous layer and without a direct connection. In one preferred embodiment, the light source is directly connected to a rectifier. The rectifier, an optional rechargeable battery, a receiver coil array or piezoelectric device, a driver circuit and a telemetry transmitter are preferably disposed together within the patient's body, apart from the treatment site. The rectifier is electrically connected to a receiver coil array/piezoelectric device and full-wave rectifies alternating current output from the receiver coil array, producing electrical current that may be used to charge the optional rechargeable battery. If a rechargeable battery is used, the power stored therein is subsequently supplied to energize the light source(s), and/or other micro-electronic circuitry mounted thereon. The receiver coil array/piezoelectric device includes at least one receiver coil and/or piezoelectric device that is energized by electromagnetic, acoustic or RF energy transmitted from an external power source disposed outside the patient's body, adjacent to the cutaneous layer, opposite the receiver coil array/piezoelectric device.

For embodiments of implantable light source 16 in which it is preferable to provide power for the light sources or other micro-electronic circuits mounted on the light source through electromagnetic coupling, as opposed to directly through leads that extend to a remote location within the patient's body, either of two types of coils can be used. One type of receiver coil comprises a plurality of turns of conductive lead, and can be located at some distance from the treatment site within the patient's body, disposed under and adjacent to a cutaneous layer. To provide electrical energy to the light source, a transmitter coil comprising a number of turns of a conductive lead that is connected to an external power supply is disposed on the outer surface of skin immediately adjacent to the receiver coil. An alternating current applied by the external power supply develops an electromagnetic field in the external transmitter coil, that couples to the receiver coil, causing a corresponding alternating current to flow in the receiver coil. This alternating current is rectified using the full wave rectifier, which may be included within the light source, or alternatively, disposed at the receiver coil.

In a related scheme, a transmitter coil comprising a ferrite core (or a core of another material having a relatively high magnetic permeability) that is generally “C”-shaped is coupled through leads to an external power supply, which supplies an alternating current to helical conductive coils that are wrapped around a ferrite core. The alternating current flowing through conductive coils develops an electromagnetic field that is coupled to a receiver coil, disposed subcutaneously opposite the transmitter coil inside the patient's body. The receiver coil also comprises a C-shaped ferrite core, around which is helically coiled a conductor, which is coupled to leads conveying electrical current to the remotely located light source that is disposed at a remote site within the patient's body. The transmitter coil and receiver coil are oriented with their respective ferrite cores aligned, so as to maximize flux linkage between the ferrite cores. These coils are highly efficient at transferring electromagnetic energy.

The implantable light sources disclosed herein can optionally include circuitry for selectively controlling the optical parameters of the light radiation provided. A desired dose, intensity, frequency, pulse duration, wavelength or waveband, power, monochromaticity, intensity modulation, and three dimensional photon distribution of light can thereby be provided by the light source at the treatment site. This would eliminate the need for supplying a large selection of implantable light therapy devices for different applications, so that the light parameters from a single device could be for example, programmed for continuous blood irradiation in vasculature of different diameters, programmed for intermittent irradiation of small portions of organ surfaces, or programmed for blood irradiation in synchrony with environmental or physiological status. In one preferred embodiment of the present invention the physiological status may be EEG, EMG, ECG, blood chemistry, viral load, body temperature, chemiluminescence, pH, pulse and/or respiration.

As used herein in the specification, and in the claims below, the term “blood chemistry” refers to the concentration, or concentrations, of any and all substances dissolved in, or comprising, the blood. Thus, in one preferred embodiment of the invention, the light parameters are determined in accordance with the concentration of gases dissolved in the blood with or without hyperoxygenating the blood. In addition to the major constituent atmospheric gases oxygen, nitrogen and carbon dioxide, concentrations of rare gases such as xenon and other noble gases, and ozone may be monitored to provide optimal illumination for therapeutic interaction with specific gases dissolved in the blood. In another preferred embodiment, light parameters are modulated in response to concentrations of additional therapeutic agents, and/or their metabolites. Thus, specific light therapy regimen may be coordinated with dosages and timing of concurrent therapies, such as hormone replacement or chemotherapy, to provide possible enhancement and synergy of beneficial effects. These options are implemented by including appropriate modulating circuitry in control module 14, coupled between power supply 12 and light source 16. The regimen of light therapy parameters determined by the control circuitry may be preselected, prior to implantation of the light source. Thus, in one embodiment of the present invention the circuitry is an on-board logic chip. Alternatively, in a preferred embodiment of the present invention, the light therapy parameters are variably determined and the implantable light source is controlled by acoustic-based, RF-based and/or magnetic-based telemetry, where the light therapy parameters are determined from a remote, external telemetry transmitter, operably coupled to an intracorporeal telemetry reciever/transciever. Such an external transmitter may be coupled to additional devices monitoring, for example, pulse, respiration and blood pressure, as in intensive care technology. Additional sensors and programs for monitoring of physiological status and/or light radiation at the site of administration may also be integrated into the implantable control circuitry or telemetry. Examples of miniature devices for monitoring and controlling the power output of intracorporeal medical devices are described in U.S. Pat. No. 5,788,717, to Mann (regarding pacemakers), U.S. Pat. Nos. 6,185,443 and 6,119,031 to Crowley (regarding endoscopic sensors and spectroscopy) and U.S. Pat. No. 6,063,108 to Salansky, et al. (regarding low level laser therapy).

Often the site of phototherapy is inconvenient or unsuitable for implantation of the light source, as in treatment of a bone lesion, delicate vascular structures or nervous and/or contractile tissue. In such cases illumination of the treatment site may be effected by a light-transmitting conduit, such as an optical fiber. Chen et al. (U.S. Pat. No. 5,445,608) and Prescott (U.S. Pat. No. 6,156,028) describe the implantation of optical fibers to conduct light to a remote, internal treatment site, however, the light source of these devices is extracorporeal.

By using a remote light source connected to optical fibers, the light source may be implanted in a convenient location, for example, within the fascia of the pectoral or axiallary region, as is common with the pulse generator component of implantable pacemaker devices. Additional potential locations are the fascia of the lumbo-sacral and femoral regions, abdominal and pleural cavities, subcutaneous adipose tissue, etc. Thus, in a preferred embodiment of the present invention, the implantable light source is in optical communication with an optical fiber 18 for propagating light emitted from the light source to a remote intracorporeal location. The optical fiber may be designed of plastic, glass or other light propagating material, and is preferably flexible, affording access to irregular and difficult-to-reach structures. In its course between the light source and the site of illumination, the optical fiber may be secured to adjacent tissue and internal surfaces via sutures, clips, adhesives, etc. Examples of suitable optical fibers are described in U.S. Pat. Nos. 5,728,092, to Doiron et al. and U.S. Pat. No. 6,004,315, to Dumont et al. Most preferably the optical fiber is a polymeric optical fiber as described by Dumont et al., having a cladded, non light-transmitting surface, which may be converted to a light diffusing site, or plurality of light diffusing sites, by removal of the cladding and roughening of the optical fiber to provide light scattering. In this manner the requirement for an additional lens, or other means for focusing the light at the treatment site is obviated.

As used herein the phrase “optical communication” refers to any and all means of substantially efficient transmission of light radiation between a light source and a substantially non-reflective recipient element.

As described above, the implantable light source of the invention may be adapted to illuminate all surfaces, or be introduced into the tissue of internal structures. Thus, illumination of hollow organs, for example, may be effected by introduction of the light source into the lumen of such organs, and, alternatively, solid organs may be treated by location of the light source external to and/or within the tissue of such organs. Additionally, and alternatively, the abovementioned optical fiber may direct light to the surfaces or tissues of internal organs. Thus, in preferred embodiments of the present invention, the implantable light source is designed, constructed and implantable so as to illuminate a lumen of a blood vessel, a lumen of at least one heart chamber and/or the lumen of an organ. Non-limiting examples of such organs are the brain, spinal canal, sinuses, middle ear, lungs, esophagus, stomach, intestines, colon, pancreas, spleen, gall bladder, appendix, liver, kidney, bladder, heart, ovary and uterus. It will be appreciated, in the context of the present invention, that therapeutic illumination of the uterus includes treatment of developing fetal tissues. The present invention is well suited for treatment of and within a gravid uterus, providing the highly localized, controllable illumination required for restriction of treatment to the target tissues, and, perhaps more importantly, for the exclusion of sensitive fetal tissues from undesired exposure. In addition, the availability of an implanted intracorporeal light source eliminates the need for repeated procedures of illumination therapy over the lengthy period of gestation.

In another preferred embodiment the light source is designed, constructed and implantable so as to illuminate the surface and/or tissue of an organ. Non-limiting examples of such organs are the eye, brain, spinal cord, sinuses, middle ear, lungs, stomach, intestines, pancreas, spleen, liver, kidney, heart, ovary, uterus, testis, prostate, bladder, endocrine and/or exocrine glands, bone, muscle and connective tissue.

Different tissues, and tissue components, exhibit characteristic maximal and optimal light absorption parameters, often limited to a rather narrow set (bandwidth) of light frequencies. Some well-known examples are the excitation spectra of chlorophyll and rhodopsin, and the characteristic UV absorption by DNA, RNA and proteins. Some specific protocols have been established for phototherapy of a number of conditions (Karu, Health Physics, 56:691-704, 1989), mostly according to empirical results, such as UV irradiation of blood for immune modulation (Schieven, G L and Ledbetter, J A., Ultraviolet radiation induces different calcium signals in human peripheral blood lymphocyte subsets. J. Immunother 1993 October; 14(3): 221-25), low power red and near red non-coherent light for healing of skin ulcers (U.S. Pat. No. 5,259,380 to Mendes et al.) and bright, visible light for shifting circadian rhythms (U.S. Pat. No. 6,135,117 to Campbell and Murphy). Both substantially coherent and non-coherent light is effective in certain of the therapy protocols. Thus, in one preferred embodiment of the present invention, the light is coherent light between 189 (ultraviolet) and 1,300 (far red) nm in wavelength. In another, more preferred embodiment, the light is non-coherent light of a plurality of wavelengths and/or wavebands between 189 and 1300. In still another embodiment, the light is non-coherent light of at least one waveband between 189 nm and 1,300 nm.

As used herein phrase “coherent light” refers to light radiation of a single wavelength, or narrow (less than 20 nm) waveband, also known as monochromatic light. Likewise, the term “non-coherent light” refers to light of a plurality of wavelengths, or wavebands encompassing at least one range of greater than 20 nm.

As in photostimulation of other tissues, phototherapy of the blood may potentially effect many cellular and non-cellular elements. Salansky et al. (U.S. Pat. No. 6,063,108) describe a range of light parameters for therapeutic illumination of fast- and slow moving erythrocytes, fibroblasts and leukocytes. Schleicher (U.S. Pat. No. 6,113,566) lists many devices and protocols for UV irradiation of blood, including catheter and indwelling venipuncture apparati, aliquot and continuous flow devices. Traditional UV blood irradiation protocols, developed by Knott (U.S. Pat. Nos. 2,308,516 and 2,309,124) claim to be effective in spite of the relatively small volumes (less than 10% of blood volume) removed, irradiated and returned to the patient. Mersch et al. (U.S. Pat. No. 5,693,049) describes an indwelling catheter device for intracorporeal illumination of the blood, intended for short term, temporary use in UV detoxification and treatment of blood borne parasitic, viral and bacterial pathogens. Chen and Swanson (U.S. Pat. No. 5,571,152) describe a microminiature light emitting bead for implantation within the vascular system, for PhotoDynamic Therapy. However, none of the prior art provides for direct, long-term intracorporeal illumination of the blood.

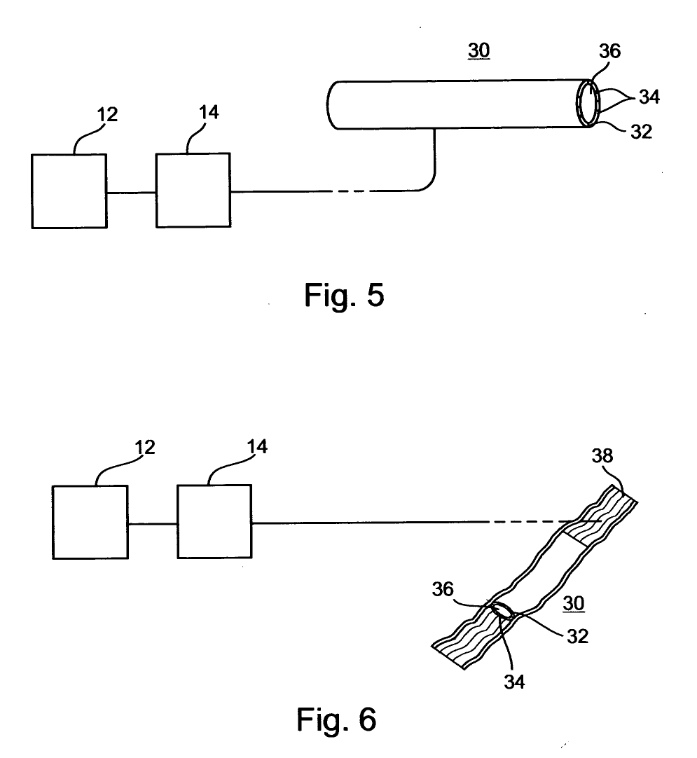

Thus, according to another aspect of the present invention there is provided a method of therapeutic illumination of blood, the method according to this aspect of the invention is effected by implanting intracorporeally in a subject in need of therapeutic illumination an implantable light source for producing light suitable for therapeutic illumination of the blood. Blood may be irradiated by direct vascular implantation of a light source bearing conductive leads connected to a remote power supply, the light source being small enough to avoid interference with normal circulatory dynamics. Alternatively, the light source may borne by an implantable tubular platform allowing blood flow therethrough, surgically introduced into the vasculature. FIG. 5 depicts tubular platform 30, bearing an array of implantable light sources 34 on it's inner surface, connected to control module 14 and power supply 12. Blood flow is provided through hollow inner bore 36. Platform 30 may also function as a stent, i.e., have sufficient structural rigidity so as to support the walls of the blood vessel.

Placement of the intravascular light sources may be effected by surgically exposing the blood vessel at or near the treatment site, introducing the light source or tubular platform bearing the light source within the lumen of the blood vessel, securing the light source or tubular platform by sutures, clips, adhesives, etc. Alternatively, the light source and/or platform may be introduced into a blood vessel from a remote, more convenient (i.e., a more superficial) location and guided to the desired implantation site using, for example, an inflatable, retractable device similar to that employed in angioplasty techniques. One such device, used for intravascular implantation of electrical pacemaker leads is disclosed by Spreigl, et al in U.S. Pat. No. 6,161,029. FIG. 6 depicts the tubular platform 30 in place within the lumen of blood vessel 38, affording circulation through the inner bore 36.

One of the advantages of blood irradiation using an intravascularly implantable light source of the present invention is the capability of selective irradiation of blood, without exposing light-sensitive endothelial tissues. By choosing an opaque material, or coating the external surface 32 of the light source-bearing tubular platform with a biocompatible, photoreflective layer, light radiation emanating from the light source is contained within the interior of the tubular platform.

Another approach to intermediate- and long-term intracorporeal irradiation of blood is to divert the circulation through a light-emitting device. Some primitive and complicated methods for external UV blood illumination devices are described in Schleicher et al (U.S. Pat. No. 6,113,566). However, today vascular surgeons commonly replace, bypass, repair, remove and graft blood vessels in cases of circulatory disease or dysfunction. Many prosthetic devices for implantation into the circulatory system are available, such as artificial valves, arteries and veins, see, for example, the vascular prostheses and connections described by Zegdi, et al (U.S. Pat. Nos. 6,187,020 and 5,893,886). Implantation of a vascular prosthesis comprising the abovementioned optical fibers for the diffusion of light to the blood flowing therethrough, in optical connection with the implantable light source according to the invention, enables intermediate- and long-term irradiation of blood for general, systemic applications (such as detoxification, anti-viral and anti-bacterial treatment) and local applications (such as brief, rhythmic illumination of blood perfusing the brain, or a portal system such as in the liver or kidney). Such a light-emitting vascular prosthesis could also be introduced in lateral anastomosing connection, parallel with a blood vessel, constituting a shunt for light therapy of the blood.

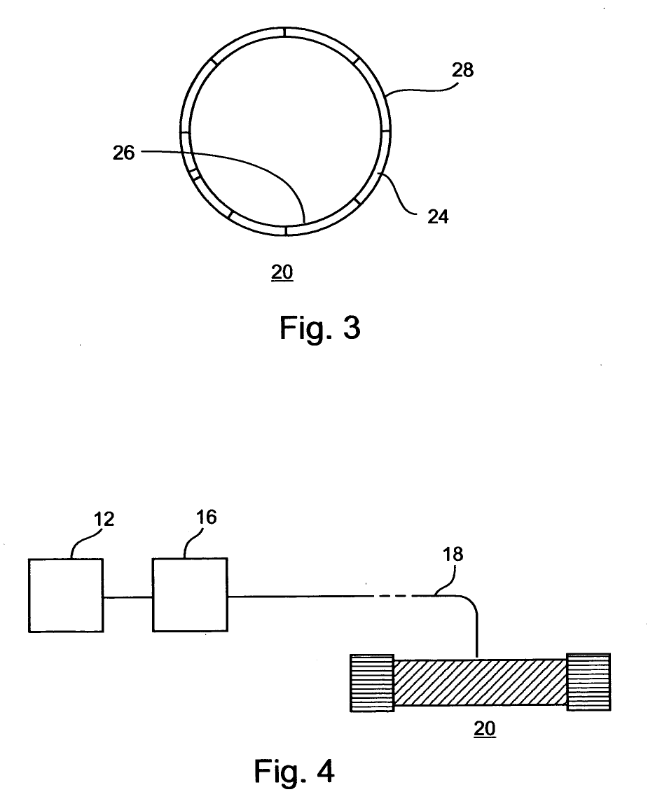

Thus, according to an additional aspect of the present invention, there is provided a device for therapeutic illumination of the blood, the device comprising an implantable vascular prosthesis capable of anastomosing connection to a blood vessel in which a lateral or terminal opening has been formed, the implantable vascular prosthesis having an internal light emitting surface. FIG. 2 depicts an implantable light emitting vascular prosthesis 20 in optical communication with implantable light source 16 via optical fiber 18. Vascular prosthesis 22 is flanked by flexible connecting sections 22, for suture or clamp-type anastomosing to adjacent blood vessels, and contains optical diffusing surfaces 24 integrated into it's internal surface 26. FIG. 3 depicts a cross sectional view of the vascular prosthesis 20, indicating a reflective outer covering 28 for preventing outward diffusion of light. FIG. 4 depicts the vascular prosthesis 20 in anastomosis with two terminal openings in a blood vessel. In the context of illumination of the blood, one widely recognized practice comprises the extracorporeal illumination of blood with UV wavelengths, enriching the blood ozone concentration and returning the ozone-rich blood to the circulation (see, for example, U.S. Pat. No. 5,591,457 to Bolton). Ozone therapy, effected extracorporeally, has been applied in treatment of viral infections, conditions which are associated with blood platelet aggregation such as arterial occlusive diseases, peripheral vascular disease; thrombotic diseases, such as coronary thrombosis, pulmonary thrombosis, arterial and venous thrombosis; circulatory disorders, such as Raynaud's disease; stroke, pre-eclampsia; hypertension and cancer. In addition, treatment of blood with ultraviolet radiation and ozone has been found to increase blood levels of prostacyclin, a substance which is known to inhibit platelet aggregation, relax peripheral blood vessels and to activate the human immune system by stimulating T-lymphocytes and monocytes, and by increasing the potential of peripheral blood mononuclear cells to proliferate. Reported effective range of ozone concentrations for the abovementioned effects is 1-100 parts per million (U.S. Pat. No. 4,632,980 to Zee, et al). Intermediate- and long-term intracorporeal illumination with suitable, ozone producing wavelengths (commonly UV, 189-400 nm) can provide a constant level of blood ozone for therapy, conceivably preferable to short-term, intermittent dosages. Thus, in one preferred embodiment of the present invention, intracorporeal illumination of the blood is combined with breathing oxygen-enriched air, increasing blood pO² and, in turn, effectively elevating the levels of circulating ozone.

Insomuch as the interaction of light radiation with blood oxygen has demonstrable beneficial effects, so may the combination of irradiation and the presence of other, less common gases, such as the noble gases. The present invention is well suited to provide intermediate- and long-term illumination of such gas-enriched blood. Thus, in another embodiment, intracorporeal illumination is combined with breathing air enriched with non-oxygen gases.

The method and device of the present invention are novel and innovative in the application, for the first time, of completely implantable illumination technology for direct phototherapy of internal tissues, including the blood. The resulting benefits include (i) intermediate and long term phototherapy of internal tissues; (ii) direct, long-term illumination of blood without effecting endothelium and neighboring tissues; (iii) provisions for intracorporeal and/or external (telemetric) power supply and control of illumination; and (iv) continuously variable, remote modulation of light therapy parameters.

It will be appreciated, in the context of the present invention, that all implantable components, and specifically intravascular elements, must be provided for use in sterile condition, free of toxicity and contamination. Thus, the entirety of abovementioned implantable light sources, control modules, power supplies, optical fibers, telemetry receivers/transceivers, light source-bearing tubular platforms, vascular prostheses and connecting elements therebetween are capable of being sterilized. Common methods of sterilization of medical devices and instruments include chemical, gas, moist- and dry heat and irradiation. Considering the delicate and complicated nature of many of the components of the present invention, a preferred method of sterilization is irradiation with ionizing radiation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of therapeutic illumination of internal organs and/or tissues, the method comprising implanting intracorporeally in a subject in need of therapeutic illumination an implantable light source for producing light suitable for therapeutic illumination.

2. The method of claim 1, wherein said implantable light source is in optical communication with an optical fiber for propagating light emitted from said light source to a remote intracorporeal location.

3. The method of claim 1, wherein said implantable light source is designed, constructed and implantable so as to illuminate a lumen of a blood vessel.

4. The method of claim 1, wherein said implantable light source is designed, constructed and implantable so as to illuminate a lumen of at least one heart chamber.

5. The method of claim 1, wherein said implantable light source is designed, constructed and implantable so as to illuminate a lumen of an organ.

6. The method of claim 5, wherein said organ is selected from the group consisting of the brain, spinal canal, sinuses, middle ear, lungs, esophagus, stomach, intestines, colon, pancreas, spleen, gall bladder, appendix, liver, kidney, bladder, heart, ovary and uterus.

7. The method of claim 1, wherein said implantable light source is designed, constructed and implantable so as to illuminate a surface of an internal organ.

8. The method of claim 7, wherein said organ is selected from the group consisting of eye, brain, spinal cord, sinuses, middle ear, lungs, stomach, intestines, pancreas, spleen, liver, kidney, heart, ovary, uterus, testis, prostate, bladder, endocrine and/or exocrine glands, bone, muscle and connective tissue.

9. The method of claim 1, wherein said light is a coherent light between 189 nm and 1,300 nm in wavelength.

10. The method of claim 1, wherein said light is a non-coherent light of a plurality of wavelengths and/or wavebands between 189 nm and 1,300 nm.

11. The method of claim 1, wherein said light is a non-coherent light of at least one waveband between 189 nm and 1,300 nm.

12. The method of claim 1, wherein said implantable light source comprises a tubular platform and a light source physically connected thereto.

13. The method of claim 12, wherein said tubular platform is transparent to light produced by said light source.

14. The method of claim 12, wherein said tubular platform is opaque to light produced by said light source.

15. The method of claim 1, wherein said implantable light source is powered by an intracorporeally implantable battery or energy transducer.

16. The method of claim 15, wherein said energy transducer is selected from the group consisting of a radiofrequency transducer, a magnetic transducer and an acoustic transducer.

17. The method of claim 1, wherein said implantable light source comprises and is powered by a battery or energy transducer integrally connected thereto.

18. The method of claim 17, wherein said energy transducer is selected from the group consisting of a radiofrequency transducer, a magnetic transducer and an acoustic transducer.

19. The method of claim 1, wherein said implantable light source is powered by telemetry.

20. The method of claim 19, wherein said telemetry is selected from the group consisting of acoustic based telemetry, radiofrequency based telemetry and magnetic based telemetry.

21. The method of claim 1, wherein said implantable light source is controlled, said control comprising determining light therapy parameters selected from a group comprising dose, intensity, frequency, pulse duration, wavelength, power, monochromaticity, intensity modulation with specific endogenous frequencies and three dimensional photon distribution.

22. The method of claim 21, wherein said light therapy parameters are preselected.

23. The method of claim 21, wherein said light therapy parameters are variably determined.

24. The method of claim 23, wherein said light therapy parameters are determined in respect to a physiological status of a subject being treated.

25. The method of claim 24, wherein said physiological status is selected from the group consisting of EEG, EMG, ECG, blood chemistry, viral load, body temperature, chemiluminescence, pH, pulse and respiration.

26. The method of claim 1, wherein said implantable light source is controlled by telemetry.

27. The method of claim 26, wherein said telemetry is selected from the group consisting of acoustic-based telemetry, radiofrequency-based telemetry and magnetic-based telemetry.

28. The method of claim 1, wherein said implantable light source is controlled by an on-board logic-chip.

29. The method of claim 1, wherein said subject is treated for a pathology selected from the group consisting of inflammations, wounds, burns, chronic ulceration's, eczema, shingles, infection, scars, skin, vascular and organ grafts, gingival irritation, oral ulcers, cellulite, arthritic conditions, muscle pain and stiffness, myofascial pain, swelling, inflammation, scarring and stiffness, sprains, strains, wounds, whiplash, repetitive strain injuries, neurological and neuromuscular conditions, jet lag, Seasonal Affective Disorder, shift work sleep disturbance, atheroslerosis following balloon angioplasty, allergic rhinitis and nasal polyposis.

30. The method of claim 1, wherein said subject is treated for a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

31. A method of therapeutic illumination of blood, the method comprising implanting intracorporeally in a subject in need of therapeutic illumination of blood an implantable light source for producing light suitable for therapeutic illumination of blood.

32. The method of claim 31, wherein said implantable light source is in optical communication with an optical fiber for propagating light emitted from said light source to a remote intracorporeal location.

33. The method of claim 31, wherein said implantable light source is designed, constructed and implantable so as to illuminate a lumen of a blood vessel.

34. The method of claim 31, wherein said implantable light source is designed, constructed and implantable so as to illuminate a lumen of at least one heart chamber.

35. The method of claim 31, wherein said light is a coherent light between 189 nm and 1,300 nm in wavelength.

36. The method of claim 31, wherein said light is a non-coherent light of a plurality of wavelengths and/or wavebands between 189 nm and 1,300 nm.

37. The method of claim 31, wherein said light is a non-coherent light of at least one waveband between 189 nm and 1,300 nm.

38. The method of claim 31, wherein said implantable light source comprises a tubular platform and a light source physically connected thereto, said tubular platform is designed and constructed to be engaged within a blood vessel.

39. The method of claim 38, wherein said tubular platform is transparent to light produced by said light source.

40. The method of claim 38, wherein said tubular platform is opaque to light produced by said light source.

41. The method of claim 31, wherein said implantable light source is powered by an intracorporeally implantable battery or energy transducer.

42. The method of claim 41, wherein said energy transducer is selected from the group consisting of a radiofrequency transducer, a magnetic transducer and an acoustic transducer.

43. The method of claim 31, wherein said implantable light source comprises and is powered by a battery or energy transducer integrally connected thereto.

44. The method of claim 43, wherein said energy transducer is selected from the group consisting of a radiofrequency transducer, a magnetic transducer and an acoustic transducer.

45. The method of claim 31, wherein said implantable light source is powered by telemetry.

46. The method of claim 45, wherein said telemetry is selected from the group consisting of acoustic based telemetry, radiofrequency based telemetry and magnetic based telemetry.

47. The method of claim 31, wherein said implantable light source is controlled, said control comprising determining light therapy parameters selected from a group comprising dose, intensity, frequency, pulse duration, wavelength, power, monochromaticity, intensity modulation with specific endogenous frequencies and three dimensional photon distribution.

48. The method of claim 47, wherein said light therapy parameters are preselected.

49. The method of claim 47, wherein said light therapy parameters are variably determined.

50. The method of claim 47, wherein said light therapy parameters are determined in respect to a physiological status of a subject being treated.

51. The method of claim 50, wherein said physiological status is selected from the group consisting of EEG, EMG, ECG, blood chemistry, viral load, body temperature, chemiluminescence, pH, pulse and respiration.

52. The method of claim 31, wherein said implantable light source is controlled by telemetry.

53. The method of claim 52, wherein said telemetry is selected from the group consisting of acoustic-based telemetry, radiofrequency-based telemetry and magnetic-based telemetry.

54. The method of claim 31, wherein said implantable light source is controlled by an on-board logic-chip.

55. The method of claim 31, wherein said subject is treated for a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

56. A device for therapeutic illumination of internal organs and/or tissues, the device comprising:
a light source for producing light suitable for therapeutic illumination;
a battery or energy transducer for powering said light source;
at least one optical fiber in optical communication with said light source for propagating light emitted from said light source to a remote intracorporeal location;
wherein said light source, said battery or energy transducer and said at least one optical fiber are designed and constructed for intracorporeal implantation.

57. The device of claim 56, designed, constructed and implantable so as to illuminate a lumen of a blood vessel.

58. The device of claim 56, designed, constructed and implantable so as to illuminate a lumen of at least one heart chamber.

59. The device of claim 56, designed, constructed and implantable so as to illuminate a lumen of an organ.

60. The device of claim 59, wherein said organ is selected from the group consisting of the brain, spinal canal, sinuses, middle ear, lungs, esophagus, stomach, intestines, colon, pancreas, spleen, gall bladder, appendix, liver, kidney, bladder, heart, ovary and uterus.

61. The device of claim 56, designed, constructed and implantable so as to illuminate a surface of an internal organ.

62. The device of claim 61, wherein said organ is selected from the group consisting of the eye, brain, spinal cord, sinuses, middle ear, lungs, stomach, intestines, pancreas, spleen, liver, kidney, heart, ovary, uterus, testis, prostate, bladder, endocrine and/or exocrine glands, bone, muscle and connective tissue.

63. The device of claim 56, wherein said light is a coherent light between 189 nm and 1,300 nm in wavelength.

64. The device of claim 56, wherein said light is a non-coherent light of a plurality of wavelengths and/or wavebands between 189 nm and 1,300 nm.

65. The device of claim 56, wherein said light is a non-coherent light of at least one waveband between 189 nm and 1,300 nm.

66. The device of claim 56, further comprising a tubular platform carrying said light source.

67. The device of claim 66, wherein said tubular platform is transparent to light produced by said light source.

68. The device of claim 66, wherein said tubular platform is opaque to light produced by said light source.

69. The device of claim 56, wherein said energy transducer is selected from the group consisting of a radiofrequency transducer, a magnetic transducer and an acoustic transducer.

70. The device of claim 56, wherein said light source is powered by telemetry.

71. The device of claim 70, wherein said telemetry is selected from the group consisting of acoustic based telemetry, radiofrequency based telemetry and magnetic based telemetry.

72. The device of claim 56, wherein said implantable light source is controlled, said control comprising determining light therapy parameters selected from a group comprising dose, intensity, frequency, pulse duration, wavelength, power, monochromaticity, intensity modulation with specific endogenous frequencies and three dimensional photon distribution.

73. The device of claim 72, wherein said light therapy parameters are preselected.

74. The device of claim 72, wherein said light therapy parameters are variably determined.

75. The device of claim 74, wherein said light therapy parameters are determined in respect to a physiological status of a subject being treated.

76. The device of claim 75, wherein said physiological status is selected from the group consisting of EEG, EMG, ECG, blood chemistry, viral load, body temperature, chemiluminescence, pH, pulse and respiration.

77. The device of claim 56, wherein said light source is controlled by telemetry.

78. The device of claim 77, wherein said telemetry is selected from the group consisting of acoustic-based telemetry, radiofrequency-based telemetry and magnetic-based telemetry.

79. The device of claim 56, wherein said light source is controlled by an on-board logic-chip.

80. The device of claim 56, indicated for treatment of a pathology selected from the group consisting of inflammations, wounds, burns, chronic ulcerations, eczema, shingles, infection, scars, skin, vascular and organ grafts, gingival irritation, oral ulcers, cellulitis, arthritic conditions, muscle pain and stiffness, myofascial pain, swelling, inflammation, scarring and stiffness, sprains, strains, wounds, whiplash, repetitive strain injuries, neurological and neuromuscular conditions, jet lag, Seasonal Affective Disorder, shift work sleep disturbance, atheroslerosis following balloon angioplasty, allergic rhinitis and nasal polyposis.

81. The device of claim 56, indicated for treatment of a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

82. The device of claim 56, wherein said light source is a non-gaseous light emitting source.

83. The device of claim 82, wherein said non-gaseous light source is selected form the group consisting of laser, light-emitting diodes, superluminous diodes and laser diodes.

84. The device of claim 56, wherein said at least one optical fiber is capable of adapting to the contour of body passages.

85. The device of claim 56, wherein said at least one optical fiber forms a bundle of optical fibers.

86. The device of claim 85, wherein said bundle of optical fibers is flexible and hence capable of adapting to contours of body passages.

87. The device of claim 85, wherein said bundle of optical fibers is engaged within a sheath.

88. The device of claim 56, wherein said at least one optical fiber forms a bundle of optical fibers designed for simultaneously delivering light to a plurality of intracorporeal locations.

89. A device for therapeutic illumination of a tissue and/or an organ, the device comprising:
an implantable vascular prosthesis capable of anastamosing connection to a blood vessel in which a lateral or terminal opening has been formed;
a light source for producing light suitable for therapeutic illumination being optically connected to said vascular prosthesis; and
an implantable battery or energy transducer for powering said light source.

90. The device of claim 89, wherein said light is a coherent light between 189 nm and 1,300 nm in wavelength.

91. The device of claim 89, wherein said light is a non-coherent light of a plurality of wavelengths and/or wavebands between 189 nm and 1,300 nm.

92. The device of claim 89, wherein said light is a non-coherent light of at least one waveband between 189 nm and 1,300 nm.

93. The device of claim 89, wherein said energy transducer is selected from the group consisting of a radiofrequency transducer, a magnetic transducer and an acoustic transducer.

94. The device of claim 89, wherein said light source is powered by telemetry.

95. The device of claim 94, wherein said telemetry is selected from the group consisting of acoustic based telemetry, radiofrequency based telemetry and magnetic based telemetry.

96. The device of claim 89, wherein said light source is controlled, said control comprising determining light therapy parameters selected from a group comprising dose, intensity, frequency, pulse duration, wavelength, power, monochromaticity, intensity modulation with specific endogenous frequencies and three dimensional photon distribution.

97. The device of claim 96, wherein said light therapy parameters are preselected.

98. The device of claim 96, wherein said light therapy parameters are variably determined.

99. The device of claim 98, wherein said light therapy parameters are determined in respect to a physiological status of a subject being treated.

100. The device of claim 99, wherein said physiological status is selected from the group consisting of EEG, EMG, ECG, blood chemistry, viral load, body temperature, chemiluminescence, pH, pulse and respiration.

101. The device of claim 89, wherein said light source is controlled by telemetry.

102. The device of claim 101, wherein said telemetry is selected from the group consisting of acoustic-based telemetry, radiofrequency-based telemetry and magnetic-based telemetry.

103. The device of claim 89, wherein said light source is controlled by an on-board logic-chip.

104. The device of claim 89, indicated for treatment of a pathology selected from the group consisting of inflammations, wounds, burns, chronic ulcerations, eczema, shingles, infection, scars, skin, vascular and organ grafts, gingival irritation, oral ulcers, cellulitis, arthritic conditions, muscle pain and stiffness, myofascial pain, swelling, inflammation, scarring and stiffness, sprains, strains, wounds, whiplash, repetitive strain injuries, neurological and neuromuscular conditions, jet lag, Seasonal Affective Disorder, shift work sleep disturbance, atheroslerosis following balloon angioplasty, allergic rhinitis and nasal polyposis.

105. The device of claim 89, indicated for treatment of a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

106. The device of claim 89, wherein said light source is a non-gaseous light emitting source.

107. The device of claim 106, wherein said non-gaseous light source is selected form the group consisting of laser, light-emitting diodes, superluminous diodes and laser diodes.

108. The device of claim 89, wherein said vascular prosthesis contains at least one optical fiber in optical connection with said light source.

109. The device of claim 108, wherein said at least one optical fiber forms a bundle of optical fibers.

110. The device of claim 109, wherein said bundle of optical fibers is flexible and hence capable of adapting to contours of body passages.

111. The device of claim 109, wherein said bundle of optical fibers is engaged within a sheath.

112. The device of claim 89, wherein said at least one optical fiber forms a bundle of optical fibers designed for simultaneously delivering light to a plurality of intracorporeal locations.

113. A device for therapeutic illumination of blood, the device comprising an implantable vascular prosthesis capable of anastamosing connection to a blood vessel in which a lateral or terminal opening has been formed, said implantable vascular prosthesis having an internal light emitting surface for light irradiation of substances in fluid motion through said prosthesis.

114. A device for therapeutic illumination of blood, the device comprising:
an implantable tubular platform allowing blood flow therethrough;
a light source for producing light suitable for therapeutic illumination being carried by said implantable tubular platform; and
an implantable battery or energy transducer for powering said light source.

115. The device of claim 114, wherein said light is a coherent light between 189 nm and 1,300 nm in wavelength.

116. The device of claim 114, wherein said light is a non-coherent light of a plurality of wavelengths and/or wavebands between 189 nm and 1,300 nm.

117. The device of claim 114, wherein said light is a non-coherent light of at least one waveband between 189 nm and 1,300 nm.

118. The device of claim 114, wherein said tubular platform is transparent to light produced by said light source.

119. The device of claim 114, wherein said tubular platform is opaque to light produced by said light source.

120. The device of claim 114, wherein said energy transducer is selected from the group consisting of a radiofrequency transducer, a magnetic transducer and an acoustic transducer.

121. The device of claim 114, wherein said light source is powered by telemetry.

122. The device of claim 121, wherein said telemetry is selected from the group consisting of acoustic based telemetry, radiofrequency based telemetry and magnetic based telemetry.

123. The device of claim 114, wherein said light source is controlled, said control comprising determining light therapy parameters selected from a group comprising dose, intensity, frequency, pulse duration, wavelength, power, monochromaticity, intensity modulation with specific endogenous frequencies and three dimensional photon distribution.

124. The device of claim 123, wherein said light therapy parameters are preselected.

125. The device of claim 123, wherein said light therapy parameters are variably determined.

126. The device of claim 125, wherein said light therapy parameters are determined in respect to a physiological status of a subject being treated.

127. The device of claim 126, wherein said physiological status is selected from the group consisting of EEG, EMG, ECG, blood chemistry, viral load, body temperature, chemiluminescence, pH, pulse and respiration.

128. The device of claim 114, wherein said light source is controlled by telemetry.

129. The device of claim 128, wherein said telemetry is selected from the group consisting of acoustic-based telemetry, radiofrequency-based telemetry and magnetic-based telemetry.

130. The device of claim 114, wherein said light source is controlled by an on-board logic-chip.

131. The device of claim 114, indicated for treatment of a pathology selected from the group consisting of inflammations, wounds, burns, chronic ulcerations, eczema, shingles, infection, scars, skin, vascular and organ grafts, gingival irritation, oral ulcers, cellulitis, arthritic conditions, muscle pain and stiffness, myofascial pain, swelling, inflammation, scarring and stiffness, sprains, strains, wounds, whiplash, repetitive strain injuries, neurological and neuromuscular conditions, jet lag, Seasonal Affective Disorder, shift work sleep disturbance, atheroslerosis following balloon angioplasty, allergic rhinitis and nasal polyposis.

132. The device of claim 114, indicated for treatment of a pathology selected from the group consisting of diabetic angiopathy, IDDM, chronic foot ulcers, ischemic heart disease, rheumatoid arthritis, autonomic vascular dystonia, atherosclerosis, a typical pneumonia, poliomyelitis and polioencephalitis, hepatitis, HIV, AIDS, influenza, common upper respiratory diseases, herpes simplex and zoster, mumps, mononucleosis, measles, porphyria, hyperbilirubinemia and parasitic infections.

133. The device of claim 114, wherein said light source is a non-gaseous light emitting source.

134. The device of claim 133, wherein said non-gaseous light source is selected form the group consisting of laser, light-emitting diodes, superluminous diodes and laser diodes.

135. The device of claim 114, wherein said tubular platform contains at least one optical fiber in optical connection with said light source.

136. The device of claim 135, wherein said at least one optical fiber forms a bundle of optical fibers.

137. The device of claim 136, wherein said bundle of optical fibers is flexible and hence capable of adapting to contours of body passages.

138. The device of claim 136, wherein said bundle of optical fibers is engaged within a sheath.

139. The device of claim 114, wherein said at least one optical fiber forms a bundle of optical fibers designed for simultaneously delivering light to a plurality of intracorporeal locations.
